(19) United States
(12) Patent Application Publication
(10) Pub. No.: US 2008/0227209 A1
(43) Pub. Date: Sep. 18, 2008

(54) Methods, Kits And Devices For Analysis Of Lipoprotein(a)

(76) Inventor: David Xing-Fei Deng, Mountain View, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/686,093

(22) Filed: Mar. 14, 2007

(51) Int. Cl. G01N 33/92

(52) U.S. Cl. 436/71

(57) ABSTRACT

Methods for optically detecting lipoproteins, in particular lipoprotein(a), in a sample. In some embodiments, the methods include contacting a sample with an associative lipophilic dye, subjecting the sample to electrophoretic separation through a separation medium, and detecting the dye. Kits comprising microchips for performing a separation of lipoproteins, including lipoprotein(a), are also provided.

### BACKGROUND

Lipoprotein(a) [Lp(a)] has been well recognized as an independent risk factor for cardiovascular diseases, including coronary heart disease and stroke (Alfthan et al. Atherosclerosis (1994) 106:9-19; Milionis et al. J Clin Pathol. (2000) 53:487-96; Fujino et al. Jpn Circ J. (2000) 64:51-6; Schreiner et al. Ann Epidemiol. (1994) 4:351-9; Zhuang et al. Chin Med J (Engl). (1993) 106:597-600; Marcovina et al. Arterioscler Thromb. (1993) 13:1037-45; Woo et al. J Clin Lab Anal. (1991) 5:335-9). Structurally, Lp(a) is a complex macromolecule containing apolipoprotein B-100, the main lipoprotein of low density lipoprotein (LDL) particles and a carbohydrate-rich, highly hydrophilic protein, apolipoprotein (a) [apo(a)], in which one molecule of apo(a) is covalently linked to one lipoprotein B-100 component by a disulfide bridge (Koschinsky et al. Curr Opin Lipidol. (2004) 15:167-74; Guevara et al. Proteins. (1992) 12:188-99).

It is the apo(a) that distinguishes Lp(a) from all other lipoprotein classes including LDL. The apo(a) is not only of high carbohydrate content (up to 30% of the protein mass), but also exhibits considerable heterogeneity in size and structure (Koschinsky et al. (2004); Peynet et al. Atherosclerosis (1999) 142:233-9; Ali et al. Hum Biol. (1998) 70:477-90). Apo(a) is formed by three distinct structural domains, each exhibiting a high degree of homology with plasminogen (Koschinsky et al. (2004); Guevara et al. (1992)). Plasminogen is formed by a protease domain and by five domains called kringles 1 through 5. Each kringle domain contains six conserved cysteine residues, which form three disulfide bonds that provide the characteristic triple loop structure of the kringles. Apo(a) contains an inactive protease domain and one copy of kringle 5 domain (both of which exhibit approximately 85% homology with the corresponding domains of plasminogen) and multiple copies of the plasminogen-like kringle 4 (K4) domain, which are similar (but not identical) to each other and can be divided into 10 distinct kringle types (K4 types 1 through 10). Their homology with plasminogen K4 ranges between 78% and 88%. One copy each of K4 type 1 and types 3 through 10 is present per apo(a) particle. However, K4 type 2 is present in a variable number of repeats (from 3 to >40), which are therefore responsible for the size heterogeneity of apo(a) and consequently of Lp(a) (Gaubatz et al. J Lipid Res. (1990) 31.603-13).

Because apo(a) is structurally highly similar to plasminogen, Lp(a) physiologically differs from LDL and is prothrombotic, which may play an important role in its pathogeneicity of coronary heart disease and stroke (Koschinsky et al. (2004); Marcovina et al. Curr Opin Lipidol. (2003) 14:361-6; Gunther et al. Stroke) 2000) 31:2437-41; Lynch et al. Pediatrics (2005) 116:447-53; Marcucci et al. J Thromb Haemosl. (2005) 3:502-7). Also, apo(a) is highly heterogeneous, although the pathophysiological significance of Lp(a) polymorphism is not fully understood. There is some evidence suggest that Lp(a) polymorphism might be an important aspect of Lp(a) as a risk factor (Peynet et al. Atherosclerosis (1999); Gaubatz et al. (1990); Ichinose et al. Biochem Biophys Res Commun. (1995) 209:372-8).

The current widely accepted method for the determination of serum Lp(a) level, immunochemical analysis, which applies antibodies against apo(a) portion of the Lp(a), cannot accurately and reproducibly assess Lp(a) level due to the highly heterogeneous nature of apo(a) (polymorphism).

It would be desirable to provide improved methods for analyzing lipoprotein(a) in order to better characterize the atherogenic risk of a patient and to obtain more information for managing patients.

### SUMMARY

Provided herein are improved kits, methods, and devices for detecting and analyzing lipoproteins, including Lp(a).

In some aspects, described herein are new approaches to quantitate Lp(a) level by staining the apoB-100 portion of Lp(a) via associative lipophilic dyes. The associative lipophilic dye(s) stain apolipoproteins, such as apoB-100, but not other serum proteins, such as, e.g., albumin and hemoglobin. In some embodiments, the associative dyes are lipophilic. Non-limiting examples of suitable dyes include 1,1′-dioctadecyl-3,3,3′,3′-tetramethylindocarbocyanine perchlorate (DiI); 3,3′-dioctadecyloxacarbocyanine perchlorate (DiO); 1,1′-dioctadecyl-3,3,3′,3′-tetramethylindodicarbocyanine perchlorate (DiD); Vybrant DiD; 1,1′-dioctadecyl-3,3,3′,3′-tetramethylindotricarbocyanine iodide (DiR); N-(4,4-difluoro-5,7-dimethyl-4-bora-3a,4a-diaza-s-indacene-3-pentanoyl)sphingosine (BODIPY® FL C5-ceramide), and sub-combinations thereof. Other examples include polymethine dyes, such as benzopylyrium polymethine (DY-630-OH).

In some embodiments, Lp(a) is separated by different migration times from other lipid particles containing apolipoproteins (VLDL, LDL and HDL) in a microchip electrophoresis system. Various separation matrices can be used, non-limiting examples of which include polyacrylamide, polydimethylacrylamide, polyethylene oxide, polyvinyl pyrrolidone, and hydroxypropyl cellulose.

Kits for use in carrying out the methods described herein are provided. In some embodiments, kits can comprise a calibration sample, such as a ladder. A ladder can include Lp(a). A ladder can include a size variant of Lp(a). A ladder can include VLDL, IDL, LDL, HDL, Lp(a), a size variant of Lp(a) and combinations thereof. Kits can include a separation channel and a micro-chip adapted for separations described herein.

The new approaches can accurately and rapidly measure Lp(a) levels in serum or plasma. By using the methods and kits described herein, lipoproteins, including Lp(a) and size-isoforms of Lp(a), can be selectively detected.

Additional advantages and novel features of the methods, compositions, devices, and kits will be set forth in part in the description which follows, and in part will become apparent to those skilled in the art upon examination of the following description, or may be learned by practice of the methods.

### BRIEF DESCRIPTION OF THE DRAWINGS

Embodiments can be more completely understood in connection with the following drawings, in which:

FIG. 1 schematically illustrates the functional components required for a chip for utilization in a kit according to some embodiments, illustrated in block diagram form.

FIG. 2 schematically illustrates an exemplary chip for utilization in a kit according to some embodiments.

FIG. 3 schematically illustrates an exemplary chip for utilization in a kit according to some embodiments.

FIG. 4 schematically illustrates an exemplary microscale electrophoresis device or chip for use in electrophoretic separation of lipoproteins including lipoprotein(a) for use in the present methods.

FIG. 5 shows an electropherogram of standards analyzed on a microfluidic chip with the bioanalyzer. FIG. 5A, FIG. 5B, FIG. 5C, and FIG. 5D show the analysis of Lp(a), HDL, human albumin, and hemoglobin standards, respectively.

FIG. 6 shows an electropherogram of samples analyzed on a microfluidic chip with the bioanalyzer. FIG. 6A shows analysis of HDL and LDL standards in the presence of whole serum. FIG. 6B shows analysis of HDL and LDL standards in the presence of whole serum and Lp(a) standard.

FIG. 7 shows an electropherogram of samples analyzed on a microfluidic chip with the bioanalyzer.

### DESCRIPTION

Before describing the present disclosure in detail, it is to be understood that this disclosure is not limited to specific compositions, method steps, or equipment, as such can vary. It is also to be understood that the terminology used herein is for the purpose of describing particular embodiments only, and is not intended to be limiting. Methods recited herein can be carried out in any order of the recited events that is logically possible, as well as the recited order of events. Furthermore, where a range of values is provided, it is understood that every intervening value, between the upper and lower limit of that range and any other stated or intervening value in that stated range is encompassed within the present disclosure. Also, it is contemplated that any optional feature of the disclosed variations described can be set forth and claimed independently, or in combination with any one or more of the features described herein.

Unless defined otherwise below, all technical and scientific terms used herein have the same meaning as commonly understood by one of ordinary skill in the art to which this disclosure belongs. Still, certain elements are defined herein for the sake of clarity.

All literature and similar materials cited in this application, including but not limited to patents, patent applications, articles, books, treatises, and internet web pages, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

The publications discussed herein are provided solely for their disclosure prior to the filing date of the present application. Nothing herein is to be construed as an admission that the present disclosure is not entitled to antedate such publication by virtue of prior invention. Further, the dates of publication provided may be different from the actual publication dates, which may need to be independently confirmed.

It must be noted that, as used in this specification and the appended claims, the singular forms “a”, “an” and “the” include plural referents unless the context clearly dictates otherwise. Thus, for example, reference to “a biopolymer” can include more than one biopolymer.

It should be noted that the term “comprising” does not exclude other elements or features. Also elements described in association with different embodiments may be combined. It should also be noted that reference signs in the claims shall not be construed as limiting the scope of the claims.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

Provided herein are improved kits, methods, and devices for detecting and analyzing lipoprotein(a).

In some embodiments, there are provided methods for analyzing lipoprotein(a) in a sample. In some embodiments, the methods can include contacting the sample with a detectable associative lipophilic dye; subjecting the sample to electrophoretic separation through a separation medium; and detecting lipoprotein(a) in the medium. The methods can include quantifying the lipoprotein(a) and can include detecting size variants of Lpa(a).

According to some embodiments, kits for optically detecting lipoprotein(a) in a sample are provided. In some embodiments, such kits comprise a chip for performing a separation of lipoprotein(a) from other lipoproteins (such as, for example, LDL, and HDL), wherein the chip comprises at least one well for receiving a sample and a separation channel coupled to the at least one well and being adapted for separating different compounds. A kit can further comprise an associative lipophilic dye capable of staining lipoprotein(a). In some embodiments, the associative lipophilic dye is characterized in that it selectively binds to lipoprotein(a) and does not detectably bind to other major serum proteins, such as albumin, or other proteins, such as hemoglobin, during a separation, such as an electrophoretic separation.

Without wishing to be bound by any particular theory, the present methods, kits and devices are advantageous for the analysis of Lp(a) and other lipoproteins, in that the associative lipophilic dyes as described herein have a high affinity for the lipoprotein particles, such as apoB-100, with little or no staining activity for other types of proteins or of nucleic acids. By mixing, e.g. a human serum sample containing Lp(a) to be measured with one or more of these dyes and subsequent electrophoretic analysis in a microfluidic chip, Lp(a) can be separated from other lipoproteins, such as high density lipoprotein (HDL) and the low density lipoprotein (LDL) sub-fractions and quantitated by fluorescent intensity of dyes bound. The methods, kits and devices enable rapid and reproducible analysis for, e.g. patient serum samples for Lp(a). In some embodiments, the methods and kits can be used to separate and detect Lp(a), Lp(a) size-variants, and other lipoproteins, such as HDL and LDL.

According to some embodiments, methods of analyzing lipoproteins, including Lp(a), in a sample are provided. The methods can comprise a step in which the lipoproteins (including Lp(a)), are separated in at least one dimension. The methods can further comprise a step in which the lipoproteins are labelled with an associative lipophilic dye. The labelling step can precede the separation step. The methods can further comprise a step of optically detecting the separated and labelled lipoproteins.

Embodiments of the present disclosure relate to methods for optically detecting lipoproteins, such as Lp(a) in a sample, wherein the methods comprise a step of labelling lipoproteins with an associative lipophilic dye such as described herein.

Embodiments of the present disclosure relate to the use of an associative lipophilic dye for optically detecting lipoproteins, such as Lp(a), Lp(a) size isoforms, and/or for the analysis of lipoprotein class distribution and/or for the analysis of HDL and/or LDL subclass patterns in a sample by labelling the lipoproteins with one or more associative lipophilic dyes as described herein.

Embodiments of the disclosure can be partly or entirely embodied or supported by one or more suitable software programs, which can be stored on or otherwise provided by any kind of data carrier and which might be executed in or by any suitable data processing unit. Software programs or routines can be preferably applied to the method of analyzing lipoproteins, e.g. in the step of detecting the labelled lipoproteins or in a step of calibrating the obtained signals or converting them into a gel-like image. For example, calibration steps according to some embodiments can be realized by a computer program, i.e. by software, or by using one or more special electronic optimization circuits, i.e. in hardware, or in hybrid form, i.e. by means of software components and hardware components.

Exemplary embodiments of kits are described herein. However, these embodiments also apply for the method of analyzing lipoproteins, for the method of optically detecting lipoproteins and for the use of associative lipophilic dyes for optically detecting lipoproteins; for the analysis of lipoprotein class distribution; for the analysis of HDL and/or LDL subclass patterns; for the analysis of Lp(a) and Lp(a) size-isoforms.

According to some embodiments of a kit, the separation channel is adapted for separating different compounds electrophoretically, chromatographically or electrochromatographically.

According to some embodiments of a kit, the separation channel is adapted for separating different compounds electrophoretically by electrophoresis selected from the group consisting of SDS polyacrylamide electrophoresis (SDS-PAGE), capillary electrophoresis and micro-channel/microfluidic channel electrophoresis.

According to some embodiments, a kit comprises a chip. The chip can comprise an element for applying an electrical field across a separation channel. According to some embodiments of the kit, the chip can comprise a material selected from the group consisting of glass, quartz, silica, silicon, and polymers.

According to some embodiments, a kit for use in carrying out the present methods comprises a separation medium. In some embodiments, the separation medium can comprise a hydrophilic polymer. Non-limiting examples of suitable hydrophilic polymers include polyacrylamide, polydimethylacrylamide, polyethylene oxide, polyvinyl pyrrolidone and polydimethylacrylamide. There are no particular limits on the polymer which can be used to effect the separation, as long as suitable performance of the separation medium can be obtained. Suitable concentration of polymer, and suitable molecular weight of the polymer in the matrix, can be determined empirically. According to some embodiments, the matrix comprises polymers having a molecular weight less than about 1000 kDa. In some embodiments, the matrix comprises polymers having a molecular weight less than about 500 kDa. In some embodiments, the matrix comprises polymers having a molecular weight less than about 300 kDa. In some embodiments, the matrix comprises polymers having a molecular weight in the range of about 50 kDa to about 500 kDa. In some embodiments, the matrix comprises polymers having a molecular weight in the range of about 100 kDa to about 300 kDa. In some embodiments, the matrix comprises polymer having a molecular weight in the range of from 150 kDa to 250 kDa.

Polymers having selected molecular weight ranges can be prepared using conventional methods. For example, stopping reagents, such as methanol, can be included at a selected concentration in a polymerization reaction mixture in order to produce a desired range of polymer molecular weight. Specified polymer preparations can be obtained commercially (e.g., from Polysciences).

According to some embodiments, a kit can include alignment dye, associative lipophilic dye, loading buffer, running buffer and other reagents for carrying out the separation.

According to some embodiments, a kit can comprise a calibration sample. According to some embodiments of the kit, the calibration sample is a “ladder”. A ladder can comprise Lp(a). A ladder can comprise a Lp(a) which comprises a size variant of apo(a).

In some embodiments of methods, kits, and devices, there are provided herein associative lipophilic dyes.

Without wishing to be bound by any particular theory, the labelling of lipoproteins can be done by the formation of ionic or non-ionic interaction between the associative lipophilic dyes as described herein and the lipoproteins to be labelled.

Without wishing to be bound by any particular theory, it is believed that lipoproteins and Lp(a) under the buffer conditions as described herein will bear negative charges. Associative lipophilic dye(s), as described herein, can be of neutral or slightly positive charge. Alignment dyes, as described below, can be hydrophilic and of negative charge. When an electrophoretic field is applied, lipoprotein-containing particles with negative charges will move from the sample wells toward the separation channels. The associative lipophilic dyes that bind to lipoprotein particles will move along with the particles, but unbound lipophilic dyes, which have no charge or slightly positive charge, will not move along with apolipoprotein particles. Once dyes bind to and move along with the lipoprotein particles, they will not be released from the particles because of the hydrophobic interaction between dyes and lipids. The gel matrix and buffer system around the particles are hydrophilic. Due to lipophilic properties of associative dye(s) as described herein, a selective labelling of lipoproteins can be achieved. In some embodiments, the associative lipophilic dye(s) as described herein are characterized in that they detectably bind to lipoproteins such as Lp(a) during a separation procedure and do not detectably bind to albumin or to hemoglobin during such separation. Non-limiting examples of such a separation procedure include electrophoresis, chromatography and electrochromatography.

By using the kits as presently described, lipoproteins can be selectively detected and analyzed, and include lipoprotein(a), size variants of Lp(a), HDL and LDL sub-class patterns of lipoproteins.

Non-limiting examples of suitable associative lipophilic dyes include 1,1′-dioctadecyl-3,3,3′,3′-tetramethylindocarbocyanine perchlorate (DiI), 3,3′-dioctadecyloxacarbocyanine perchlorate (DiO), 1,1′-dioctadecyl-3,3,3′,3′-tetramethylindodicarbocyanine perchlorate (DiD), Vybrant DiD, 1,1′-dioctadecyl-3,3,3′,3′-tetramethylindotricarbocyanine iodide (DiR), N-(4,4-difluoro-5,7-dimethyl-4-bora-3a,4a-diaza-s-indacene-3-pentanoyl)sphingosine (BODIPY® FL C5-ceramide), and polymethine dyes, such as, e.g., benzopylyrium polymethine DY-630-OH (Dyomics). In some embodiments, combinations of 2, 3, 4, or more of such dyes can be used.

In some embodiments, a combination of 1,1′-dioctadecyl-3,3,3′,3′-tetramethylindodicarbocyanine perchlorate (DiD) and N-(4,4-difluoro-5,7-dimethyl-4-bora-3a,4a-diaza-s-indacene-3-pentanoyl)sphingosine (BODIPY® FL C5-ceramide) can be used and gives enhanced sensitivity in Lp(a) analysis as compared to the use of one dye. The molar ratio of the two dyes can range from 0.1 to 10, for example. In some embodiments, a molar ratio of 1:1 can be used.

In some embodiments, a combination of 1,1′-dioctadecyl-3,3,3′,3′-tetramethylindodicarbocyanine perchlorate (DiD), N-(4,4-difluoro-5,7-dimethyl-4-bora-3a,4a-diaza-s-indacene-3-pentanoyl)sphingosine (BODIPY® FL C5-ceramide) and benzopylyrium polymethine DY-630-OH can be used and gives enhanced sensitivity in Lp(a) analysis as compared to the use of one dye. The molar ratio of the three dyes can vary. In some embodiments, a molar ratio of 1:1:1 can be used.

In some embodiments, the present disclosure provides an associative lipophilic dye containing a polymethine with the general formula I. The substituted derivatives of indole, heteroindole, pyridine, chinoline or acridine of the general formula I can be used as associative lipophilic dyes for the optical marking of lipoproteins. Polymethines having the general formula I are described in U.S. Pat. No. 6,750,346 which is incorporated herein by reference in its entirety.

In some embodiments, there are provided herein associative lipophilic dyes which contain a non-symmetrical polymethine comprising a substituted ω-(benz[b]pyran-4-ylidene)alk-1-enyl) unit of the general formula I

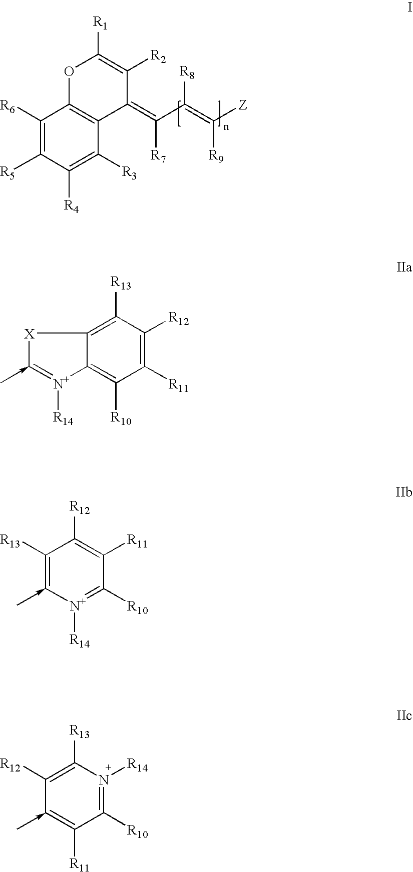

and wherein
X is selected from the group consisting of O, S, Si, N-alkyl and C(alkyl)₂,
n is 0, 1, 2 or 3,
R¹ to R¹⁴ are independently selected from the group consisting of hydrogen, alkyl, alkoxy, cycloalkyl, linear or branched alkenyl, cycloalkenyl, aryl, heteroaryl, heterocycle, hydroxy, carboxyl, amine, alkyl-substituted amine and cyclic amine and/or two or more fragments in ortho-position to each other, for example R¹⁰ and R¹¹ or R⁴, R⁵ and R⁶, together form another cycloalkyl ring or ring system, heterocyclic ring or ring system, heteroaryl ring system or aromatic ring or ring system.

At least one of the substituents R¹ to R¹⁴ can also be a solubilising or ionisable or ionised substituent like cyclodextrine, sugar, SO₃⁻, PO₃²⁻, COO⁻, or NR₃⁺ which determines the hydrophilic properties of these dyes. Such a substituent may be bound to the marker dye by means of a spacer group. For example, said solubilizing or ionisable group is bound via an aliphatic or heteroaliphatic group.

In some embodiments, at least one of the substituents R¹ to R¹⁴ can be a reactive group which is capable of reacting with a lipoprotein to form a covalent or non-covalent bond. Such a substituent can also be bound to the dye by means of a spacer group. Examples for such reactive groups are selected from the group consisting of an N-hydroxy-succinimidester group, a maleimide group and a phosphoamidite group.

According to some embodiments, R¹ to R¹⁴ are independently selected form the group consisting of hydrogen, chlorine, bromine, and an aliphatic or mononuclear aromatic group, each having at most 12 carbon atoms which may contain as a substituted group in addition to carbon and hydrogen up to 4 oxygen atoms and 0, 1 or 2 nitrogen atoms or a sulfur atom or a sulfur and a nitrogen atom or represent an amino function, having a nitrogen atom to which there is bound hydrogen or at least one substituent having up to 8 carbon atoms, said substituent being selected from the group consisting of carbon, hydrogen and up to two sulfonic acid groups.

According to some embodiments, any of the groups R¹ to R¹⁴ is aliphatic and contains from 1 to 6 carbon atoms.

In some embodiments, R¹ is a substituent which has a quaternary C-atom in α-position relative to the pyran ring. Examples for such substituents are t-butyl (—C(CH₃)₃), phenyl and adamantyl (—C₁₀H₁₅/tricyclo[3.3.1.1^{3,7}]decyl). It is particularly preferred that R¹=—(CH₃)₃.

In some embodiments, R², R³, R⁴, R⁶, R⁷, R⁸, R⁹, R¹⁰, R¹¹, R¹² and/or R¹³ is hydrogen.

In some embodiments, R⁵ is an amine or alkyl-substituted amine. It is particularly preferred that R⁵=—N(CH₂CH₃)₂.

In some embodiments, R⁴ and R⁵ form a saturated, partially saturated or unsaturated, substituted or un-substituted heterocyclic ring, preferably a six-membered heterocyclic ring containing one or more heteroatoms, preferably one or more nitrogen atoms, more preferably one nitrogen atom. Most preferably, the nitrogen atom of the heterocyclic ring corresponds to R⁵ and/or is substituted, e.g., by an ethyl group. It is further preferred that the heterocyclic ring contains one double bond.

In some embodiments, R⁴, R⁵ and R⁶ form a saturated, partially saturated or unsaturated, substituted or un-substituted bicyclic ring system, preferably a ten-membered bicyclic ring containing one or more heteroatoms, preferably one or more nitrogen atoms, more preferably one nitrogen atom. Most preferably, the nitrogen atom of the heterocyclic ring corresponds to R⁵. It is further preferred that the bicyclic ring system is saturated and/or unsubstituted.

In some embodiments, R¹⁴ is a hydroxyl- and/or carboxyl-substituted or unsubstituted alkyl. Examples for such substituents are —(CH₂)₃—OH, —(CH₂)₅—COOH, and —CH₃.

According to some embodiments, X is a carbon atom. The carbon atom is preferably substituted, e.g., by one or two alkyl groups such as methyl or ethyl. Most preferably, X is —C(CH₃)₂.

According to some embodiments, Z has the general formula IIa.

According to some embodiments, n is 1.

According to some embodiments, R¹ is —C(CH₃)₃, R² is hydrogen, R³ is hydrogen, R⁴ is hydrogen, R⁵ is —N(CH₂CH₃)₂, R⁶ is hydrogen, R⁷ is hydrogen, R⁸ is hydrogen, R⁹ is hydrogen, R¹⁰ is hydrogen, R¹ is hydrogen, R¹² is hydrogen, R¹³ is hydrogen, R¹⁴ is —(CH₂)₃—OH, Z has the general formula IIa, X is C(CH₃)₂ and/or n is 1.

According to some embodiments, R¹ is —C(CH₃)₃, R² is hydrogen, R³ is hydrogen, R⁴ is hydrogen, R⁵ is —NH₂, R⁶ is hydrogen, R⁷ is hydrogen, R⁸ is hydrogen, R⁹ is hydrogen, R¹⁰ is hydrogen, R¹¹ is hydrogen, R¹² is hydrogen, R¹³ is hydrogen, R¹⁴ is —(CH₂)₃—OH, Z has the general formula IIa, X is —C(CH₃)₂ and/or n is 1.

According to some embodiments, R¹ is —C(CH₃)₃, R² is hydrogen, R³ is hydrogen, R⁴ is hydrogen, R⁵ is —N(CH₂CH₃)₂, R⁶ is hydrogen, R⁷ is hydrogen, R⁸ is hydrogen, R⁹ is hydrogen, R¹⁰ is hydrogen, R¹¹ is hydrogen, R¹² is hydrogen, R¹³ is hydrogen, R¹⁴ is —CH₃, Z has the general formula IIa, X is —C(CH₃)₂ and/or n is 1.

According to some embodiments, R¹ is C₆H₅, R² is hydrogen, R³ is hydrogen, R⁴ is hydrogen, R⁵ is —N(CH₂CH₃)₂, R⁶ is hydrogen, R⁷ is hydrogen, R⁸ is hydrogen, R⁹ is hydrogen, R¹⁰ is hydrogen, R¹¹ is hydrogen, R¹² is hydrogen, R¹³ is hydrogen, R¹⁴ is —(CH₂)₃—OH, Z has the general formula IIa, X is —C(CH₃)₂ and/or n is 1.

According to some embodiments, the polymethine of the general formula I is selected from one of the following compounds III to IX. Non-limiting examples of counter-ions to the compounds having the general formula I and especially to compounds III to IX are F⁻, Cl⁻, Br⁻, I⁻, ClO₄⁻ or BF₄⁻.

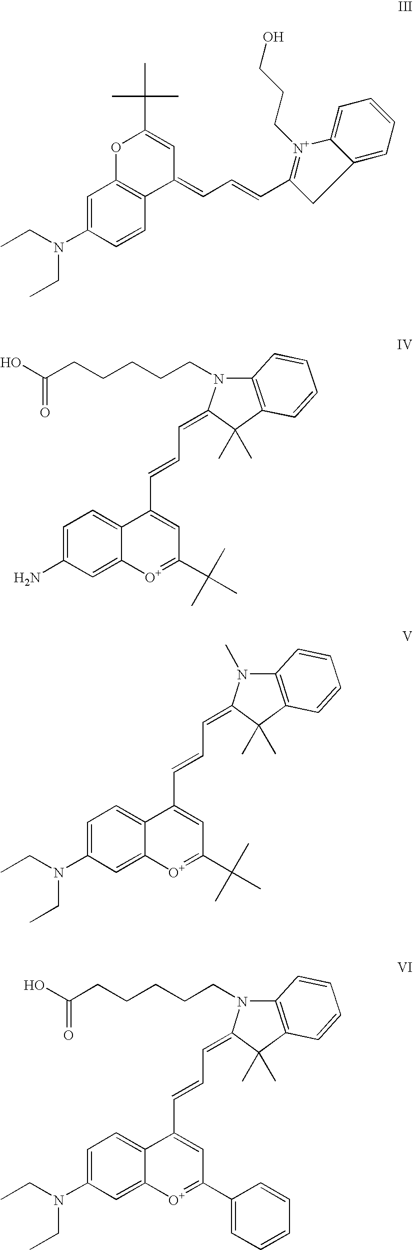

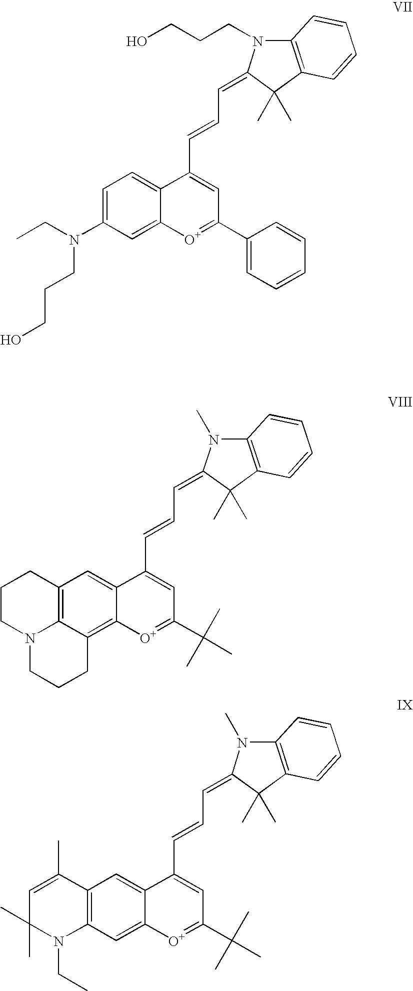

As used herein, the term “alkyl” means a linear or branched saturated aliphatic hydrocarbon group having a single radical and 1-10 carbon atoms. Examples of alkyl groups include methyl, propyl, isopropyl, butyl, n-butyl, isobutyl, sec-butyl, tert-butyl and pentyl. A branched alkyl means that one or more alkyl groups such as methyl, ethyl or propyl, replace one or both hydrogens in a CH₂ group of a linear alkyl chain. The term “lower alkyl” means an alkyl of 1-3 carbon atoms.

The term “alkoxy” means an “alkyl” as defined above connected to an oxygen radical.

The term “cycloalkyl” means a non-aromatic mono- or multicyclic hydrocarbon ring system having a single radical and 3-12 carbon atoms. Exemplary monocyclic cycloalkyl rings includes cyclopropyl, cyclopentyl and cyclohexyl. Exemplary multicyclic cycloalkyl rings include adamantyl and norbornyl.

The term “alkenyl” means a linear or branched aliphatic hydrocarbon group containing a carbon-carbon double bond having a single radical and 2-10 carbon atoms.

A “branched” alkenyl means that one or more alkyl groups such as methyl, ethyl or propyl replace one or both hydrogens in a —CH₂ or —CH═ linear alkenyl chain. Exemplary alkenyl groups include ethenyl, 1- and 2-propenyl, 1-, 2- and 3-butenyl, 3-methylbut-2-enyl, 2-propenyl, heptenyl, octenyl and decenyl.

The term “cycloalkenyl” means a non-aromatic monocyclic or multicyclic hydrocarbon ring system containing a carbon-carbon double bond having a single radical and 3 to 12 carbon atoms. Exemplary monocyclic cycloalkenyl rings include cyclopropenyl, cyclopentenyl, cyclohexenyl or cycloheptenyl. An exemplary multicyclic cycloalkenyl ring is norbornenyl.

The term “aryl” means a carbocyclic aromatic ring system containing one, two or three rings which may be attached together in a pendent manner or fused, and containing a single radical. Exemplary aryl groups include phenyl, naphthyl and acenaphthyl.

The term “heterocyclic” or “heterocycle” means cyclic compounds having one or more heteroatoms (atoms other than carbon) in the ring, and having a single radical. The ring may be saturated, partially saturated or unsaturated, and the heteroatoms may be selected from the group consisting of nitrogen, sulfur and oxygen. Examples of saturated heterocyclic radicals include saturated 3 to 6-membered hetero-monocyclic groups containing 1 to 4 nitrogen atoms, such as pyrrolidinyl, imidazolidinyl, piperidino, piperazinyl; saturated 3- to 6-membered hetero-monocyclic groups containing 1 to 2 oxygen atoms and 1 to 3 nitrogen atoms such as morpholinyl; saturated 3- to 6-membered hetero-monocyclic groups containing 1 to 2 sulfur atoms and 1 to 3 nitrogen atoms, such as thiazolidinyl. Examples of partially saturated heterocyclic radicals include dihydrothiophene, dihydropyran and dihydrofuran. Other heterocyclic groups can be 7 to 10 carbon rings substituted with heteroatoms such as oxocanyl and thiocanyl. When the heteroatom is sulfur, the sulfur can be a sulfur dioxide such as thiocanyldioxide.

The term “heteroaryl” means unsaturated heterocyclic radicals, wherein “heterocyclic” is as previously described. Exemplary heteroaryl groups include unsaturated 3 to 6-membered hetero-monocyclic groups containing 1 to 4 nitrogen atoms, such as pyrrolyl, pyridyl, pyrimidyl and pyrazinyl; unsaturated condensed heterocyclic groups containing 1 to 5 nitrogen atoms, such as indolyl, quinolyl and isoquinolyl; unsaturated 3 to 6-membered hetero-monocyclic groups containing an oxygen atom, such as furyl; unsaturated 3 to 6-membered hetero-monocyclic groups containing a sulfur atom, such as thienyl; unsaturated 3 to 6-membered hetero-monocyclic groups containing 1 to 2 oxygen atoms and 1 to 3 nitrogen atoms, such as oxyzolyl; unsaturated condensed heterocyclic groups containing 1 to 2 oxygen atoms and 1 to 3 nitrogen atoms, such as benzoxazolyl; unsaturated 3 to 6-membered hetero-monocyclic group containing 1 to 2 sulfur atoms and 1 to 3 nitrogen atoms, such as thiazolyl; and unsaturated condensed heterocyclic group containing 1 to 2 sulfur atoms and 1 to 3 nitrogen atoms, such as benzothiazolyl. The term “heteroaryl” also includes unsaturated heterocyclic radicals, wherein “heterocyclic” is as previously described, in which the hetero-cyclic group is fused with an aryl group, in which aryl is as previously described. Exemplary fused radicals include benzofuran, benzdioxole and benzothiophene.

As used herein, the term “heterocyclic C₁₋₄ alkyl”, “heteroaromatic C₁₋₄ alkyl” and the like refer to the ring structure bonded to a C₁₋₄ alkyl radical.

As used herein, the term “ring” “ring system” includes cycloalkyl, cycloalkenyl, aryl, heteroaryl or heterocycle.

All of the cyclic ring structures disclosed herein can be attached at any point where such connection is possible, as recognized by one skilled in the art.

As used herein, the term “halogen” includes fluoride, bromide, chloride, or iodide.

In accordance with some aspects of the present disclosure, a separation medium is used in carrying out the methods described herein, which medium comprises a polymer matrix, a buffering agent, and a detergent. An associative lipophilic dye can also be included in the buffering agent and/or the medium. A variety of polymer matrices can be used, including cross-linked and/or gellable polymers. In some embodiments, non-crosslinked polymer solutions are used as the polymer matrix. Non-crosslinked polymer solutions that are suitable for use in the presently described methods, compositions, and kits have been previously described for use in separation of nucleic acids by capillary electrophoresis, see, e.g., U.S. Pat. Nos. 5,264,101, 5,552,028, 5,567,292, and 5,948,227, each of which is hereby incorporated herein by reference. Such non-crosslinked or “linear” polymers provide advantages of ease of use over crosslinked or gelled polymers. In particular, such polymer solutions, because of their liquid nature, are more easily introduced into capillary channels and are ready to be used, whereas gelled polymers typically require a cross-linking reaction to occur while the polymer is within the capillary.

In some embodiments, there are provided herein non-crosslinked polymer solutions which comprise polyacrylamide polymer. The polyacrylamide polymer can be a polydimethylacrylamide polymer solution which may be neutral, positively charged or negatively charged. Without being bound to a particular theory of operation, it is believed that the polymer solutions have a dual function in the systems described herein. One function is to provide a matrix, which retards the mobility of larger species moving through it relative to smaller species. Another function of these polymer solutions is to reduce or eliminate electroosmotic flow of the materials within a capillary channel. It is believed that the polymer solutions do this by adsorbing to the capillary surface, thereby blocking the sheath flow, which characterizes electroosmotic flow.

In some embodiments, the non-crosslinked polymer is present within the separation medium at a concentration of between about 0.01% and about 30% (w/v). Different polymer concentrations can be used depending upon the type of separation that is to be performed, e.g., the nature and/or size of the lipoproteins to be characterized, the size of the capillary channel in which the separation is being carried out, and the like. Suitable concentrations can be determined empirically. In some embodiments, the polymer is present in the separation medium at a concentration of from about 0.01% to about 20%, between about 0.01% and about 10%, between about 0.1% and about 10%, or between 1% and about 5%.

The average molecular weight of the polymer within the polymer solutions of the separation medium can vary. Suitable molecular weights can be determined empirically. In some embodiments, the polymer solutions used in accordance with the present disclosure have an average molecular weight in the range of from about 1 kDa (kiloDaltons) to about 5,000 kDa, between about 1 kDa and about 1000 kDa, between about 100 kDa and about 1000 kDa, between about 50 kDa and about 500 kDa, between about 100 kDa and about 500 kDa, or between about 150 kDa and about 250 kDa.

In addition to incorporation of a polymer solution, separation media used herein can also comprise a buffering agent, a detergent, and an associative lipophilic dye.

In general, the buffering agent and detergent can be provided at concentrations which optimize separation efficiencies of lipoproteins.

Detergents incorporated into separation media can be selected from any of a number of detergents that have been described for use in electrophoretic separations. In some embodiments, anionic detergents can be used. Alkyl sulfate and alkyl sulfonate detergents can be used, non-limiting examples of which include sodium octadecyl sulfate, sodium dodecylsulfate (SDS) and sodium decylsulfate. Suitable concentrations can be determined empirically. In some embodiments, the separation medium comprises such a detergent at a concentration of between about 0.02% and about 0.15% or between about 0.03% and about 0.1%. In some embodiments, the separation medium comprises such a detergent at a concentration of between about 0.01 mM and about 1 mM, between about 0.1 mM and about 1 mM, or between about 0.1 mM and 0.3 mM.

The buffering agent can be selected from any of a number of different buffering agents. Non-limiting examples of suitable buffers include tris, tris-glycine, HEPES, TAPS, MOPS, CAPS, MES, Tricine, Tris-Tricine, combinations of these, and the like. A separation according to methods of the present disclosure can be performed at a pH in the range of from about 7 to about 8, at a pH in the range of from about 7.3 to about 7.7, or at pH of about 7.5. In some embodiments, when using a detergent at the above-described concentrations in a separation medium, the buffering agent can be provided at a concentration between about 10 mM and about 300 mM, for example.

In some embodiments, a sample containing lipoproteins for which separation is desired can be combined with a detergent, which can be present in any suitable concentration. For example, it can be in an amount of from about 0.10 to about 0.20 mM, in an amount of from about 0.125 to about 0.175 mM, or in an amount of about 0.15 mM.

In some embodiments, methods and kits according to the present disclosure comprise one or more associative lipophilic dyes. Associative lipophilic dyes as described herein, can be used in optical, in particular fluorescence optical qualitative and quantitative determination methods for electrophoresis, chromatography and electrochromatography. In some embodiments, these dyes are characterized as being neutral. In some embodiments, these dyes are characterized as having a slightly positive charge. Thus, in addition to the foregoing components, a separation medium can also comprise an associative lipophilic dye or other detectable labeling group, which associates with lipoproteins, such as Lp(a), that are to be characterized/separated. This enables the detection of lipoproteins as they are traveling through the separation medium. As used herein, an “associative lipophilic dye” refers to a detectable labeling compound or moiety, which associates with a class of molecules of interest, e.g., lipoproteins, preferentially with respect to other molecules in a given mixture. In some embodiments, an associative lipophilic dye can be present within the separation medium at a concentration between about 0.1 μM and 1 mM, and in some embodiments, between about 1 μM and about 20 μM.

Associative lipophilic dyes as described herein can be injected into a separation channel, such as a microchannel, together with the sample to be analyzed, or added before or after the sample has been injected. Associative lipophilic dyes can be contained in the separation medium.

An alignment dye can also be injected into a microchannel together with the sample. Alignment dyes can be selected that rapidly traverse the separation channel, and are used to align or normalize the migration times of the macromolecules under analysis. For example, the peak due to an alignment dye can be used as a “to” value. An alignment dye can be hydrophilic and negatively charged. Non-limiting examples of suitable alignment dyes include Alexa 700 (InVitrogen) and Dyomic-676 (Dyomics, Germany).

According to some embodiments, methods and kits disclosed herein comprise a separation medium for performing a separation of macromolecular species such as lipoproteins, including lipoprotein(a). Examples of appropriate materials for inclusion in this separation medium comprise polyacrylamide, polydimethylacrylamide, polyethylene oxide and/or polyvinylpyrrolidone, and polydimethylacrylamide (PDMA) matrix. In some embodiments, the separation medium can comprise PEO (polyethylene oxide), for which the MW may be in the range of from 10 kDa to 200 kDa, and, in some embodiments, in the range of from about 20 kDa to about 60 kDa. Optionally, the medium may also comprise a denaturing agent such as N-methylurea.

Methods and kits as described herein can be employed in various electrophoretic techniques. Non-limiting examples of electrophoretic techniques include SDS poly-acrylamide gel electrophoresis (SDS-PAGE), capillary electrophoresis, and micro-channel/microfluidic channel electrophoresis.

According to some embodiments, the separation channel is adapted for separating different compounds electrophoretically, chromatographically or electrochromatographically. E.g., a chip for performing an electrophoretic separation comprises a base substrate comprising a main surface, wherein a channel is formed in said main surface of said base substrate in at least one direction.

A chip may comprise an element for applying an electric field across the separation channel or the medium. The electric field is applied across said separation channel by turning on a voltage. Application of an electric field effects a separation of the compounds in the sample.

In some embodiments, kits and devices used for microfluidic-channel electrophoresis can comprise a micro-channel chip having a network of micro-channels that serve as paths for the migration of fluid sample volumes. A single sample volume or many sample volumes may be run on the same micro-channel chip simultaneously. The micro-channel chip can be loaded into a device, such as a bioanalyzer for molecular assays (e.g., an Agilent 2100 bioanalyzer), which provides a network of microelectrodes onto the chip wells, thus supplying the necessary voltages and currents for the separation of the sample volume components. Micro-channel chip electrophoresis can provide higher resolution, smaller sample volume sizes, shorter analysis times, and reduced sample handling over traditional capillary electrophoresis. An example of this type of electrophoresis is described in U.S. Pat. No. 6,042,710, which is hereby incorporated herein by reference in its entirely.

When used for microfluidic-channel electrophoresis, the chip can have electrodes and a substrate which comprises a planar body structure in which grooves are fabricated to define capillary channels when overlaid with a cover element, also typically planar in structure. Exemplary substrates materials include, e.g. glass, quartz, silica, silicon, polymers, e.g. plastics like polydimethylsiloxanes (PDMS), polymethylmethacrylate (PMMA), poly-urethane, polyvinylchloride (PVC), polystyrene, polysulfone, polycarbonate, polytetrafluoro-ethylene (Teflon™), and a variety of others that are well known in the art. Substrates may take a variety of shapes or forms, including tubular substrates, e.g. polymer or fused silica capillaries, or the like. In some aspects, however, the substrate comprises a planar body structure in which grooves are fabricated to define capillary channels when overlaid with a cover element, also typically planar in structure. Examples of such planar capillary systems are described in U.S. Pat. No. 5,976,336 incorporated herein by reference in its entirety. A separation medium is employed in the micro-channels formed in the substrate to bring about the separation of sample components passing through the micro-channels under the influence of an electric field induced across the medium by the electrodes.

Capillary channels also can be of a variety of different shapes in cross-section, including tubular channels, rectangular channels, rhomboid channels, hemispherical channels or the like, or even more arbitrary shapes such as may result from less precise fabrication techniques, e.g. laser ablation. Typically, the shape of a capillary channel will vary, depending upon the substrate type used and the method of propagation. For example, in typical fused silica capillaries, the capillary channel can be tubular. In systems employing planar substrates, channels can comprise either a rhomboid, rectangular or hemispherical cross sectional shape, depending upon the substrate material and method of fabrication of the channels.

A variety of manufacturing techniques are well known in the art for producing micro-fabricated channel systems. For example, where such devices utilize substrates commonly found in the semiconductor industry, manufacturing methods regularly employed in those industries are readily applicable, e.g. photolithography, wet chemical etching, chemical vapour deposition, sputtering, electroforming, etc. Similarly, methods of fabricating such devices in polymeric substrates are also readily available, including injection molding, embossing, laser ablation, LIGA techniques and the like. Other useful fabrication techniques include lamination or layering techniques, used to provide intermediate micro-scale structures to define elements of a particular micro-scale device.

In some embodiments, the capillary channels will have an internal cross-sectional dimension, e.g. width, depth, or diameter, of between about 1 μm and about 500 μm, or between about 10 μm to about 200 μm.

In some aspects, planar micro-fabricated devices employing multiple integrated micro-scale capillary channels can be used. Briefly, these planar micro-scale devices employ an integrated channel network fabricated into the surface of a planar substrate. A second substrate is overlaid on the surface of the first to cover and seal the channels, and thereby define the capillary channels.

Chips provided herein can comprise one or more analysis channels or separation channels or separation flow paths and comprises additional channels connecting the analysis channel to multiple different sample reservoirs. These reservoirs are generally defined by apertures disposed in the second overlaying substrate, and positioned such that they are in fluid communication with the channels of the device. A variety of specific channel geometries can be employed to optimise channel layout in terms of material transport time, channel lengths and substrate use. Examples of such micro-scale channel network systems are described in detail in WO 98/49548, U.S. Pat. Nos. 6,475,364; 6,235,175; 6,153,073; 6,068,752; 5,976,336; and U.S. application Ser. No. 60/060,902, which are all incorporated herein by reference in their entireties.

Introduction of the separation medium into a capillary channel or micro-channel may be as simple as placing one end of the channel into contact with the medium and allowing the medium to wick into the channel. Alternatively, vacuum or pressure may be used to drive the medium solution into the capillary channel. In integrated channel systems such as those used in chip electrophoresis, the separation medium is typically placed into contact with a terminus of a common micro-channel, e.g. a reservoir disposed at the end of a separation channel, and slight pressure is applied to force the polymer into all of the integrated channels.

In some embodiments, there are provided methods which can be performed electrophoretically, and which can comprise the following steps:

injecting the sample into a chip, wherein the chip comprises at least one well for receiving the sample, and a separation channel coupled to the at least one well and being adapted for separating different compounds; and

applying an electric field across the channel to move the sample through the channel.

A sample containing lipoproteins for which separation is desired is preferably placed in one end of the separation channel and a voltage gradient is applied along the length of the channel. As the sample components are electrokinetically transported down the length of the channel and through the medium disposed therein, those components are resolved. The separated components are then detected at a point along the length of the channel, typically near the terminus of the separation channel distal to the point at which the sample was introduced.

Detection of separated species can be carried out using a conventional fluorescent detection system. Such a detection system can be operated for detection of fluorescence of the associative lipophilic dye. Typically, such systems utilize a light source capable of directing light energy at the separation channel as the separated species are transported past. The light source typically produces light of an appropriate wavelength to activate the labelling group. Fluorescent light from the labelling group is then collected by appropriate optics, e.g. an objective lens, located above, below or adjacent the capillary channel, and the collected light is directed at a photometric detector, such as a photodiode or photomultiplier tube. The detector is typically coupled to a computer, which receives the data from the detector and records that data for subsequent storage and analysis.

Before a sample comprising a plurality of unknown species is analyzed, the measurement set-up can be calibrated using a calibration sample. The calibration sample can be selected from a large variety of different calibration samples comprising a set of compounds of different size such as, for example, SRM 1951b—Lipids in Frozen Human, Serum, Level I (NIST, Gaithersburg, Md., USA), Ultra HDL calibrator vial, 1 ml (Genzyme Diagnostics, West Malling Kent, ME, UK), Human HDL, 10 mg; Human LDL, 5 mg; Human Ox. LDL, 2 mg; Human Lp(a), 0.1 mg (all available at BTI, Biomedical Technologies, Inc., MA, USA), AutoHDL/LDL Calibrator, 3 ml; HDL Standard, 15 ml (both available at Eco-Scientific, Rope Walk, Thrupp, Stroud, UK), Lipid Control Levels 1, 2 and 3 (all available at Polymedco, Inc., Cortland Manor, N.Y., USA), Low total cholesterol, TCh@50 mg/dL, LRC LEVEL 1; Normal total cholesterol, TCh@165-180 mg/dL, TG<100 mg/dL, LRC LEVEL 2; Elevated total cholesterol, TCh@265, TG@230; LRC LEVEL 3; High Density Lipoprotein, HDL @ 50, LRC LEVEL 4 (all available at Solomon Park Research Laboratories, Kirkland, Wash., USA), and HDL Reference Pools ID 204 (TV (SD) 60.1 (0.7) mg/dL), ID 205 (TV (SD) 30.5 (0.8) mg/dL), ID 301 (TV (SD) 49.5 (1.2) mg/dL), ID 303 (TV (SD) 50.6 (1.4) mg/dL), ID 305 (TV (SD) 30.8 (0.8) mg/dL), ID 307 (TV (SD) 40.5 (0.9) mg/dL) (all available at Centers for Disease Control and Prevention Atlanta, Ga. 3034, USA; note: pools may be prepared according to the Lipid Standardization Program (LSP)). A ladder may comprise an Lp(a) particle which contains a particular isoform of apo(a). A ladder may comprise a plurality of apo(a) isoforms. Marcovina et al. ((1993) Biochem Biophys Res Comm 191:1192-1196) have designated 34 different apo(a) isoforms, numbered 1-34. A ladder can comprise a plurality of Lp(a) particles comprising one or more of these different isoforms.

A ladder can be used as a calibration sample. A ladder is a calibration sample comprising a plurality of well-known components. The name “ladder” is due to the fact that the calibration peak pattern looks like a ladder of peaks related to the various components. Because the set of calibration peaks looks like a ladder, calibration samples are often referred to as “ladders”.

In some embodiments of the present disclosure, ladders comprising species covalently labelled with fluorescence tags may be employed. When the species of the calibration sample are stimulated with incident light, the tags attached to the species emit fluorescence light. Calibration samples or “ladders” comprising a marker that fluoresces at a first wavelength, and a set of labelled fragments that emit fluorescent light at a second wavelength may also be employed. In some embodiments, none of the species in a ladder are covalently labelled with fluorescent tags, but are non-covalently associated with an associative lipophilic dye as described herein, before or during application of the ladder to the separation medium.

After the fluorescent peak pattern of the calibration sample has been acquired, a sample of interest can be analyzed. In some embodiments, in order to allow for an alignment with the calibration peak pattern, a certain concentration of an associative lipophilic dye and a certain concentration of the largest labelled ladder fragment (such as, e.g., Lp(a)) can be added to a sample of interest, followed by separation and analysis. In some embodiments, in order to allow for an alignment with the calibration peak pattern and between samples, an alignment dye can be added. Compounds of the sample of interest can be separated, and the sample bands obtained at the separation column's outlet can be analyzed.

In some embodiments, an associative lipophilic dye emits fluorescent light of a first wavelength, whereas the covalently labelled species of a calibration sample emits fluorescence light of a second wavelength, which is different from the first wavelength. Some of the available ladders comprise two or more different fluorescence dyes adapted for emitting fluorescence light of two or more different wavelengths. Correspondingly, there exist fluorescence detection units adapted for simultaneously tracking fluorescence intensity at two or more wavelengths.

Methods for peak pattern calibration can be utilized. Exemplary methods are disclosed in European patent application 1 600 771 which is incorporated herein by reference in its entirety.

In the following, some embodiments of methods of analyzing lipoprotein(a) are described.

Samples to be analyzed can be subjected to preliminary treatment before they undergo the analysis. This preliminary treatment may, for example, consist of a purification and/or enrichment steps. For example, ultracentrifugation and immuno-affinity chromatography on anti-Apo(a)sepharose may be used to enrich for lipoprotein(a) (see, e.g., Gaubatz et al. (1986) Methods Enzymol. 129:167-185). In some embodiments, immuno-affinity for apolipoprotein B-100 can be used to enrich for lipoproteins.

The present disclosure provides devices and systems for use in carrying out the above described protein characterization methods. The devices of the present disclosure can include a supporting substrate which includes a separation zone into which is placed the separation buffer. A sample that is to be separated/characterized is placed at one end of the separation zone and an electric field is applied across the separation zone, causing the electrophoretic separation of the lipoproteins within the sample. The separated lipoproteins are then separately detected by a detection system disposed adjacent to and in sensory communication with the separation zone.

The devices and reagents of the present disclosure can be used in conjunction with an overall analytical system that controls and monitors the operation and analyses that are being carried out within the microfluidic devices and utilizing the reagents described herein. In particular, the overall systems typically include, in addition to a microfluidic device or capillary system, an electrical controller operably coupled to the microfluidic device or capillary element, and a detector disposed within sensory communication of the separation zone or channel of the device.

In the area of material flow, the materials to be examined, possibly in addition to the reagents required for the corresponding test such as the associative lipophilic dyes, can be fed to a microchip. Thereafter, these materials on the microchip are moved or transported, e.g. by means of electrical forces, pressure sources, thermal sources or the like. The feed and/or the movement of materials may be brought about by means of a suitable electronic control.

The test results can be detected on a suitable detection point of the chip or microchip. Detection can take place by means of optical detection, e.g. by a laser diode in conjunction with a photoelectric cell, or a mass spectrometer, which may be connected or by means of electrical detection. The resultant optical measurement signals can then fed to a signal-processing system and thereafter to an analysis unit (e.g. a suitable microprocessor) for interpretation of the measurement results.

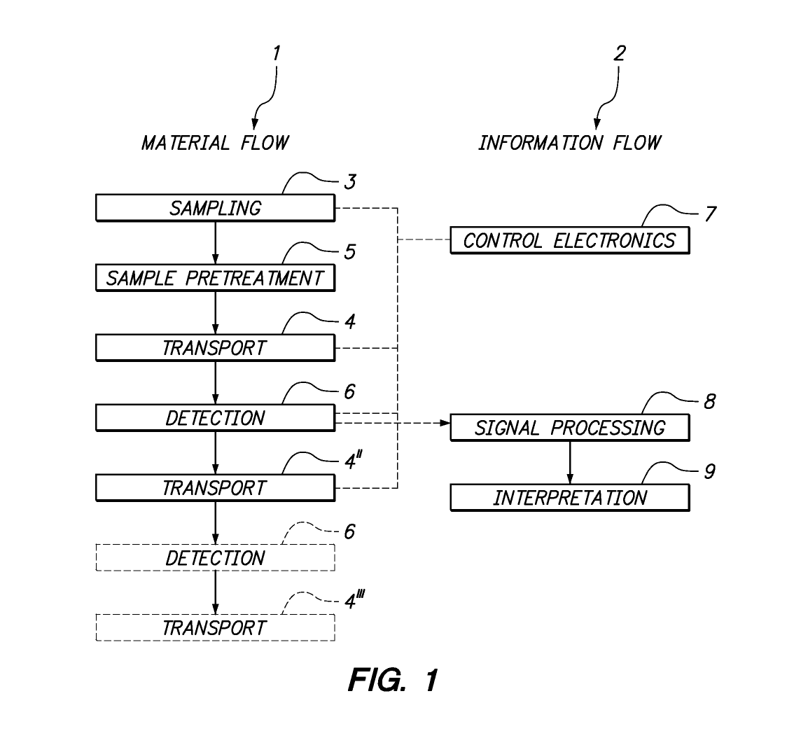

The operational components typically used for a chip used in methods in kits as described herein are schematically illustrated in FIG. 1. These are mainly subdivided into the components relating to a material transport or flow 1, and those which represent the information flow 2 arising upon execution of a test. Material flow 1 typically includes sampling operations 3, as well as optional operations for treatment or pre-treatment 5 of the materials to be examined. Furthermore, a sensor system 6 can be employed to detect the results of a test and, optionally, to monitor the material flow operations, so that adjustments can be made in controlling material flow using the control system. One example of the control mechanism is shown as control electronics 7. Data obtained in the detection operation 6 and 6′ is transferred typically to the signal processing 8 operation so that the detected measurement results can be analyzed. An objective in the design of such microchip systems is the provision of function units/modules corresponding to the above-mentioned functions and the establishment of suitable interfaces between individual modules. By means of a suitable definition of these interfaces, it is possible to achieve a high degree of flexibility in adapting the systems to various microchips or experimental arrangements. Furthermore, on the basis of such a strictly modular system structure, it is possible to achieve an extensive level of compatibility between various microchips and/or microchip systems.

Initially, in the area of material flow, the materials to be examined (possibly in addition to the reagents required for the corresponding test) are fed to the microchip 3. Thereafter, these materials on the microchip are moved or transported, e.g., by means of electrical forces 4. Both the feed and the movement of materials are brought about by means of a suitable electronic control 7, as indicated by means of the dotted line. In this example, the materials can be subjected to an optional preliminary treatment 5, before they undergo the test as such. Essentially, both the material quantity (quantity) and the material speed (quality) can be determined by means of the transportation as described. In particular, precise adjustment of material quantity enables precise metering of individual materials and material components. Furthermore, the latter processes can advantageously be controlled by means of electronic control 7.

The test results can be detected on a suitable detection point of the microchip 6. Detection advantageously takes place by means of optical detection, e.g. a laser diode in conjunction with a photoelectric cell, a mass spectrometer, which may be connected, or by means of electrical detection. The resultant optical measurement signals are then fed to a signal-processing system 8, and thereafter to an analysis unit (e.g. suitable microprocessor) for interpretation 9 of the measurement results.

Following the above-mentioned detection 6, there is the option of implementation, as indicated by the dotted line, of further test series or analyses or separation of materials, e.g., those in connection with various test stages of a chemical test cycle which is, overall, more complicated. For this purpose, materials can be transported onwards on the microchip after the first detection point 6, and to a further detection point 6′. There, the procedure theoretically defined according to stages 4″ and 6 is performed. Finally, the materials are fed, after termination of all reactions/tests, to a material drain (not illustrated here) by means of a concluding transport cycle or collection cycle 4′″.

Further incentives for miniaturization in the field of chemical analysis include the ability and desirability to minimize the distance and time over which materials are transported. In particular, the amount of time and distance required to transport materials between the sampling of the materials and the respective detection point of any chemical reaction that has taken place shall be minimized. Separation of materials can be achieved rapidly and individual components can be separated with a higher degree of resolution than has been possible in conventional systems. Furthermore, micro-miniaturized laboratory systems enable a considerably reduced consumption of materials, particularly reagents, and a far more efficient intermixing of the components of materials. An exemplary apparatus for the operation of a microfluidic device, i.e. a microchip laboratory system for chemical processing or analysis, is described in WO 00/78454 which is incorporated herein by reference in its entirety.

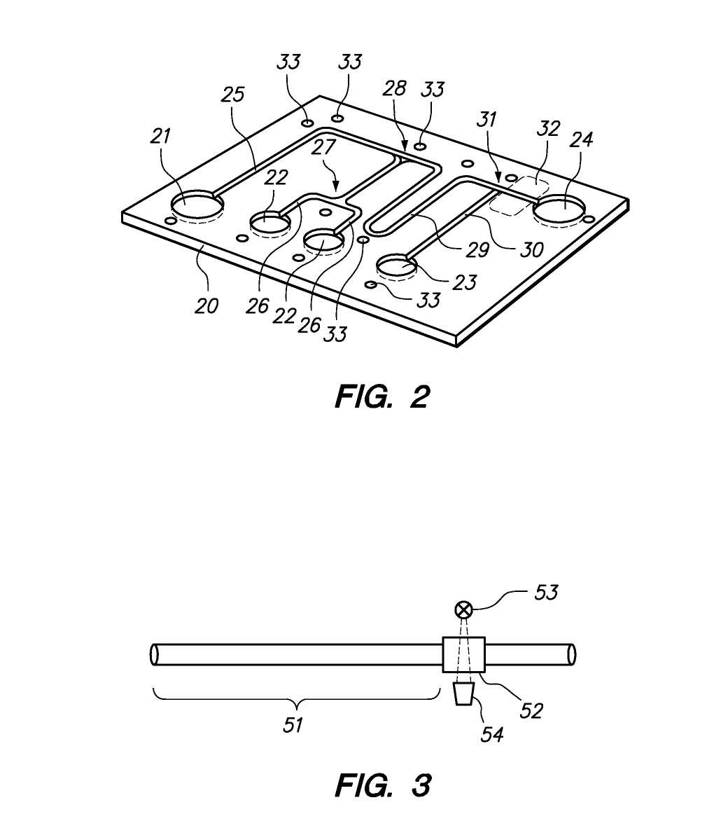

FIG. 2 shows some embodiments of laboratory microchips or chips which are suitable for utilization in a kit or method according to the present disclosure. On the upper side of a substrate 20, microfluidic structures are provided, through which materials are transported. Substrate 20 may, for example, be made up of glass or silicon, in which context the structures may be produced by means of a chemical etching process or a laser etching process. Alternatively, such substrates may include polymeric materials and be fabricated using known processes such as injection molding, embossing and laser ablation techniques. Typically, such substrates are overlaid with additional substrates in order to seal the conduits as enclosed channels or conduits.

For sampling of the material to be examined (hereinafter called the “sample material”) onto the microchip, one or several recesses 21 can be provided on the microchip, to function as reservoirs for the sample material. In performing a particular exemplary analysis or test, the sample material can be initially transported along a transport duct or channel 25 on the microchip. In this example, transport channel 25 is illustrated as a V-shaped groove for convenience of illustration. In some embodiments, the microfluidic substrates comprise scaled rectangular (or substantially rectangular) or circular-section conduits or channels.

Recesses 22 can fulfil the function of reagent and/or sample material reservoirs. In this example, two different materials could readily be manipulated. By means of corresponding transport conduits 26, these are initially fed to a junction point 27, where they intermix and, constitute the product ready to use. At a further junction 28 this reagent meets the material sample to be examined, in which the two materials will also intermix.

The material formed then passes through a conduit section 29 which may have a meandering geometry which functions to achieve artificial extension of the distance available for reaction between the material specimen and the reagent. In a further recess 23 configured as a material reservoir, in this example, there is contained a further reagent which is fed to the already available material mix at a further junction point 31.

Area 32 (or measurement zone) of the transport duct comprises a detector (e.g., a contactless detector means) which can be located above or below area 32. After the material has passed through the above-mentioned area 32, it is fed to a further recess 24 which represents a waste reservoir or material drain for the waste materials which have been produced, overall, in the course of the reaction.

On the microchip there are provided recesses 33 which act as contactless surfaces for application of electrodes and which in turn enable the electrical voltages, and even high voltages, required for connection to the microchip for operation of the chip. Alternatively, the contacting for the chips can also take place by means of insertion of a corresponding electrode point directly into the recesses 21, 22, 23 and 24 provided as material reservoirs. By means of a suitable arrangement of electrodes 33 along transport conduits 25, 26, 29 and 30 and a corresponding chronological or intensity-related harmonization of the applied fields, it is then possible to achieve a situation in which the transportation of individual materials takes place according to a precisely dictated time/quantity profile.

In pressure-driven transport of materials within the microfluidic structure, it is typically necessary to make recesses 33 such that corresponding pressure supply conduits closely and sealably engage them so as to make it possible to introduce a pressurized medium, for example in inert gas, into the transport conduits, or apply an appropriate negative pressure.

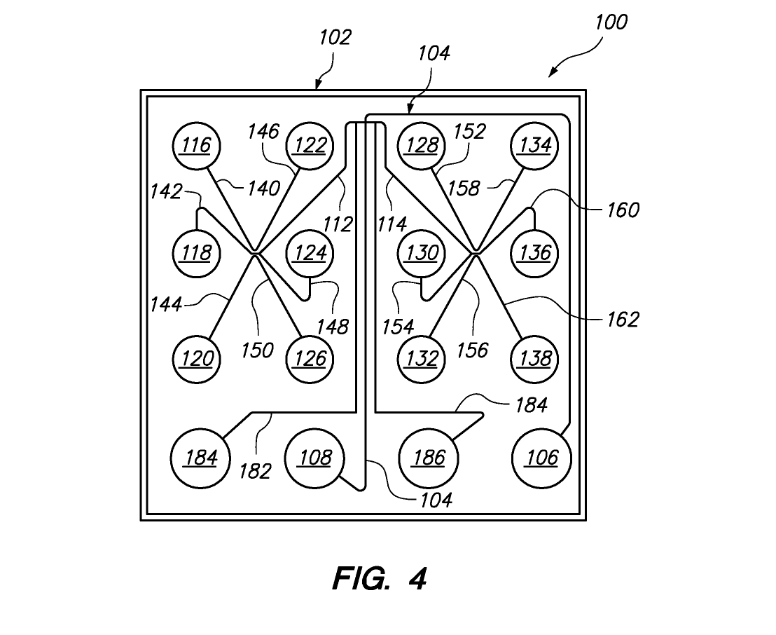

FIG. 3 shows an exemplary measurement set-up for separating and analyzing a fluid sample comprising a plurality of different sample compounds. Each of the sample compounds is characterized by an individual migration time required for travelling through a separation flow path 51. The separation flow path 51 might, e.g., be an electrophoresis flow path, a chromatography flow path or an electric chromatography flow path. At the outlet of the separation flow path, a detection cell is located. The detection cell might e.g. be implemented as a fluorescence detection cell 52 comprising a light source 53 and a fluorescence detection unit 54. The fluorescence detection cell 52 is adapted for detecting sample bands of fluorescence-labelled species as a function of time.

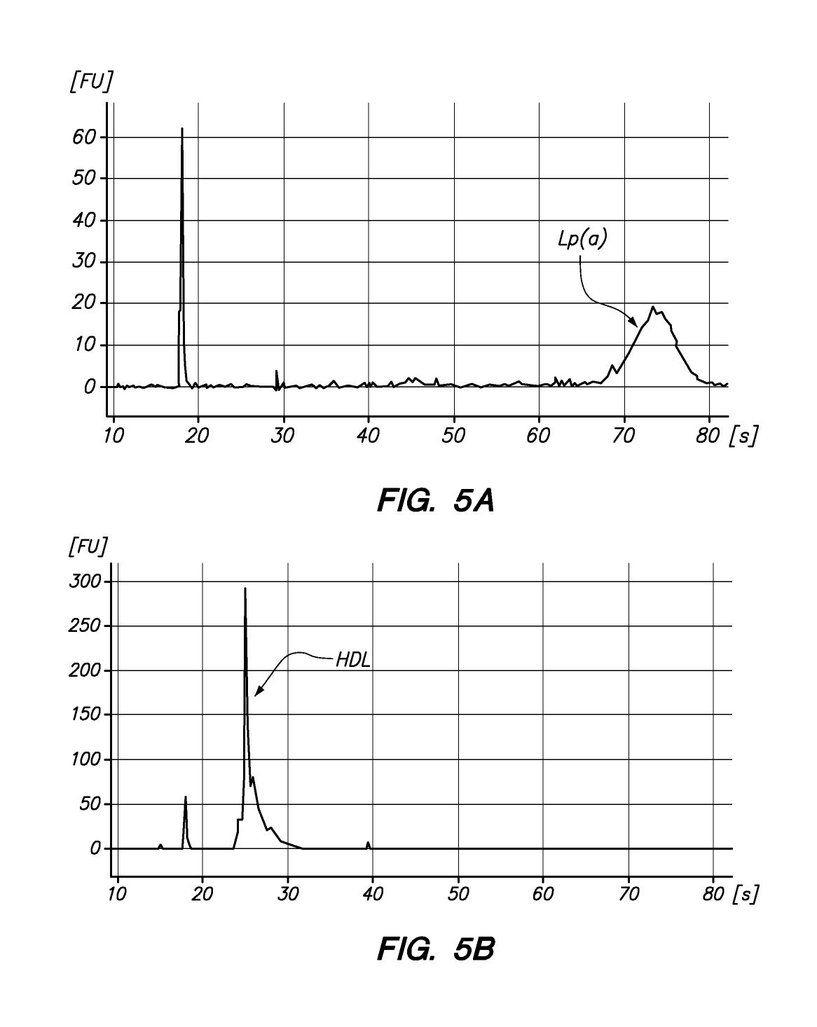

FIG. 4 shows another example of channel geometry of a chips according to the present disclosure. In operation, sample materials are placed into one or more of the sample reservoirs 116-138. A first sample material, e.g., disposed in reservoir 116, is then loaded by electrokinetically transporting it through channels 140 and 112, and across the intersection with the separation channel 104, toward load/waste reservoir 186 through channel 184. Sample is then injected by directing electrokinetic flow from buffer reservoir 106 through analysis channel 104 to waste reservoir 108, while pulling back the sample in the loading channels 112 and 114 at the intersection. While the first sample is being separated in analysis channel 104, a second sample, e.g., that disposed in reservoir 118, is preloaded by electrokinetically transporting it into channels 142 and 112 and toward the load waste reservoir 184 through channel 182. After separation of the first sample, the second sample is then loaded across the intersection with analysis channel 104 by transporting the material towards load/waste reservoir 186 through channel 184.

Exemplary methods of electrophoretically separating macromolecular species, as well as compositions, systems, devices or chips useful in carrying out such methods are described in U.S. Pat. No. 6,042,710 which is incorporated herein by reference in its entirety.

Non-limiting examples of devices for operating a microchip with a microfluid structure for chemical, physical and/or biological processing are described in European patent application 1 360 992 and international patent application WO 00/78454 which are both also incorporated herein by reference in their entirety.

In some embodiments, a separation medium used in a chip can be characterized by a value of theoretical plates. Theoretical plate numbers (N) can be calculated based on a peak obtained after injection of a standard Lp(a). An exemplary standard is human Lp(a), having molecular wt. of 536 kD (catalogue no. BT-917, Biomedical Technologies Inc.).

The calculation is based on measurement of the full width at half maximum (Δt_{1/2}) of the standard peak by using the expression:

N=5.54×(t/Δt_{1/2})²

where t is the migration time of the standard Lp(a). Theoretical plates per second (N/s) can be calculated by dividing the theoretical plate numbers (N) by the migration time. The migration time of the standard can be normalized by subtracting the migration time of an alignment dye.

In some embodiments, in chips as described herein, the separation medium is characterized by having an N/s value of at least 1, 2, 3, 4, 5, 6, 7, 8, 9, or 10 sec⁻¹. In some embodiments, in chips as described herein, the separation medium is characterized by having an N/s value in the range of between 3 sec⁻¹ and 100 sec⁻¹, 5 sec⁻¹ and 500 sec⁻¹, 5 sec⁻¹ and 100 sec⁻¹, 5 sec⁻¹ and 50 sec⁻¹, 7 sec⁻¹ and 20 sec⁻¹, or 8 sec⁻¹ and 10 sec⁻¹. The following is a sample calculation using data obtained as described in the Examples.

5.54×(60 sec/6 sec)²/60 sec=9.2 sec⁻¹

In some embodiments, an LDL standard (such as, e.g., L7914 available from Sigma-Aldrich) can be separated along with (or in parallel with) the Lp(a) standard. In some embodiments, an LDL standard can be separated in a separate microchannel in a chip and simultaneously with the Lp(a) standard. In some embodiments, in chips described herein, the separation medium is characterized in that there is baseline separation between the standard LDL and the standard Lp(a). In some embodiments, the separation can be at least 5 sec, at least 10 sec, at least 15 sec, at least 20 sec, at least 30 sec, at least 40 sec, or at least 60 sec. In some embodiments, the separation can be from about 5 sec to about 60 sec, from 10 sec to about 40 sec, or from about 20 sec to about 30 sec.

In some embodiments, in analyzing a sample using chips as described herein, the separation medium is characterized such that there is baseline separation between LDL and Lp(a). In some embodiments, the separation can be at least 5 sec, at least 10 sec, at least 15 sec, at least 20 sec, at least 30 sec, at least 40 sec, or at least 60 sec. In some embodiments, the separation can be from about 5 see to about 60 sec, from 10 sec to about 40 sec, or from about 20 sec to about 30 sec.

As described herein, the present disclosure provides kits for use in carrying out the described methods. Generally, such kits include a capillary or microfluidic device as described herein. The kits can comprise the various components of the separation buffer, e.g., the non-crosslinked polymer sieving matrix, detergent, buffering agent and the lipophilic dye. These components may be present in the kit as separate volumes of preformulated buffer components, which may or may not be pre-measured, or they may be provided as volumes of combined preformulated reagents up to and including a single combination of all of the reagents, whereby a user can simply place the separation buffer directly into the microfluidic device. In addition to the buffer components, kits according to the present disclosure also optionally include other useful reagents, such as molecular weight standards, as well as tools for use with the devices and systems, e.g., instruments which aid in introducing buffers, samples or other reagents into the channels of a microfluidic device.

In the kit form, the reagents, device and instructions detailing the use thereof can be provided in a single packaging unit, e.g., box or pouch, and sold together. Provision of the reagents and devices as a kit provides the user with ready-to-use, less expensive systems where the reagents are provided in more convenient volumes, and have all been optimally formulated for the desired applications, e.g., separation of LDL from Lp(a).

Some embodiments are subsequently to he illustrated in more detail by means of the following examples.

### EXAMPLE 1

In the following, an embodiment of methods and kits according to some embodiments of the present disclosure for the separation of lipoproteins is shown. This example demonstrates binding of an associative lipophilic dye to HLD and to Lp(a), but not to albumin or to hemoglobin.

The sample buffer contained the following reagents:
200 mM TAPS(N-Tris(hydroxymethyl)methyl-3-aminopropanesulfonic acid), pH 7.5 (Sigma, Deisenhofen, Germany, Catalogue no. T5130).
30 μM Vybrant DiD (InVitrogen, Catalogue no. V-22887) as the associative lipophilic dye.
1 μM Alexa 700 (Invitrogen—Molecular Probes, USA) as the alignment marker.
0.15 mM SDS (sodium dodecyl sulfate) (Sigma).
   The separation medium contained the following reagents:
2% Poly(N,N-dimethyl acrylamide) with MW 210 kDa and Mn 82 kDa (Polysource, Montreal, Canada. Catalogue no. P6173F4-DMA). (MW is the weighted average of molecular weight (mass); Mn is the number average molecular weight.)
200 mM TAPS(N-Tris(hydroxymethyl)methyl-3-aminopropanesulfonic acid), pH 7.5 (Sigma).
0.15 mM SDS (Sigma).
0.15 μM dye V02-04064 (Dyomics) as the dye for focusing the detector.
   Lp(a) standard and serum standard:
The Human Lp(a) standard (0.68 mg/mL), was purchased from Biomedical Technologies Inc. (Stoughton, Mass.) (BTI) (Catalogue no. BT-917), and was isolated from single polymorph donor.
The HDL standard was purchased from BTI.
The human albumin standard (Sigma) was diluted in PBS to 5 g/dL.
The hemoglobin standard (Sigma) was diluted in PBS into 14 g/dL
   The following assay protocol was used:

### Sample Staining

Take 2 μL of the Lp(a) standard, and mix with 48 μL of sample buffer.
Take 2 μL of the HDL standard, and mix with 48 μL of sample buffer.
Take 1 μL of the diluted albumin standard and mix with 49 μL of sample buffer.
Take 1 μL of the diluted hemoglobin standard and mix with 49 μL of sample buffer.

### Chip Priming

Place chip on primer station.
Label each chip.
Add 10 μL of the separation medium to the matrix well.
Pressurize the well for 1 min.
Fill the other two matrix wells with 10 μL separation medium.
Add 7 μL of each diluted sample (diluted in sample buffer) to each of the 12 sample wells.

### Chip Running

Place chip into instrument and start run.

For performing the assay, the Agilent 2100 Bioanalyzer (Agilent Technologies, USA) was used (with an applied voltage of 1100 volts).

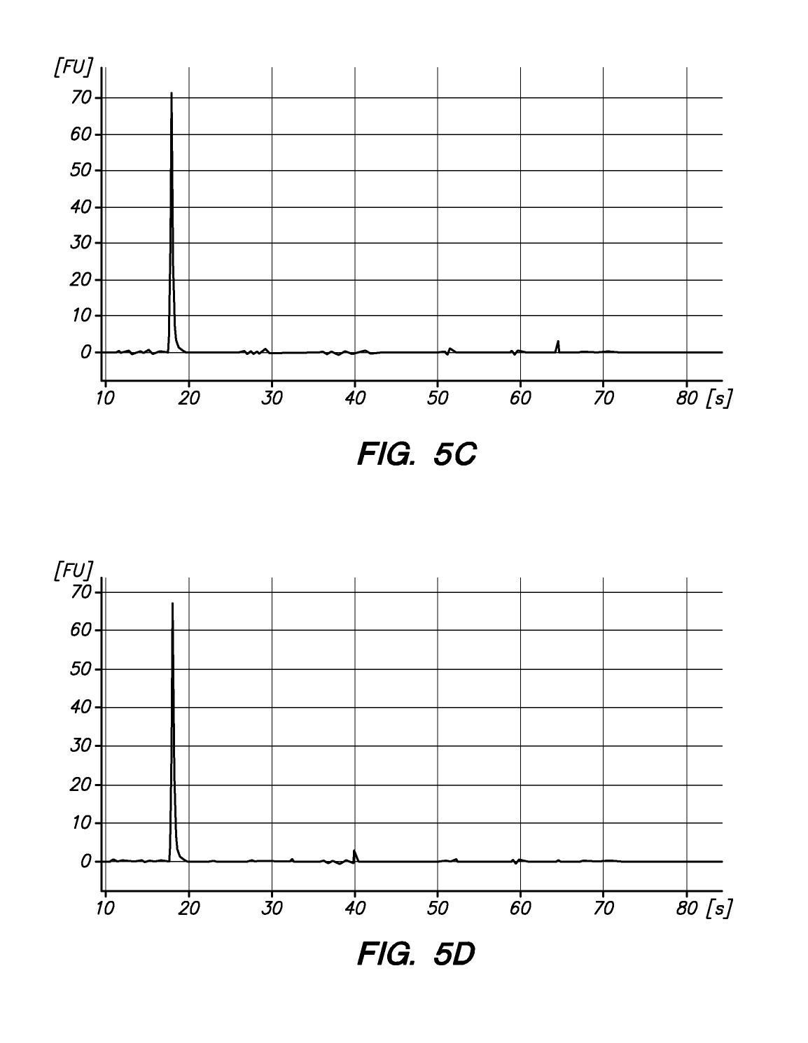

FIG. 5 shows an electropherogram of the samples analyzed on a microfluidic chip with the Bioanalyzer. FIG. 5A, FIG. 5B, FIG. 5C, and FIG. 5D show the analysis of Lp(a), HDL, human albumin, and hemoglobin standards, respectively. The associative lipophilic dye bound to Lp(a) and to HDL, but not to albumin nor to hemoglobin.

### EXAMPLE 2

In the following, an embodiment of methods and kits according to some embodiments of the present disclosure for the separation of lipoproteins is shown:

The sample buffer contained the following reagents:
200 mM TAPS (N-Tris(hydroxymethyl)methyl-3-aminopropanesulfonic acid), pH 7.5 (Sigma, Deisenhofen, Germany, Catalogue no. T5130).
30 μM Vybrant DiD (InVitrogen, Catalogue no. V-22887) as the associative lipophilic dye.
1 μM Alexa 700 (Invitrogen—Molecular Probes, USA) as the alignment marker.
0.15 mM SDS (sodium dodecyl sulfate) (Sigma).
   The separation medium contained the following reagents:
2% Poly(N,N-dimethyl acrylamide) with MW 210 kDa (Polysource, Catalogue no. P6173F4-DMA).

200 mM TAPS, pH 7.5 (Sigma).
0.15 mM SDS (Sigma).
0.15 μM dye V02-04064 (Dyomics).
   Lp(a) standard and serum standard:
The Human Lp(a) standard (0.68 mg/mL), was purchased from Biomedical Technologies Inc. (Stoughton, Mass.) (BTI) (Catalogue no. BT-917), and was isolated from single polymorph donor.
The HDL and LDL standards were purchased from BTI.
The whole serum standard (from pooled serum) was purchased from QuantiMatrix (Redondo Beach, Calif.).
   The following assay protocol was used:
Take 1 μL of serum standard and mix with 49 μL of sample buffer (no spike sample).
Take 1 μL of serum standard plus 1 μL of Lp(a) standard and mix with 48 μL of sample buffer (Lp(a) spiked sample).
Take 1 μL of the HDL standard, and the LDL standard, and separately mix each with 49 μl of sample buffer.
Place chip on primer station.
Label each chip.
Add 10 μL of the separation medium to the matrix well.
Pressurize the well for 1 min.
Fill the other two matrix wells with 10 μL separation medium.
Add 10 μl of diluted serum standard in sample buffer to the standard well.
Add 7 μL of each diluted sample (diluted in sample buffer) to each of the 12 sample wells.
Place chip into instrument and start run.

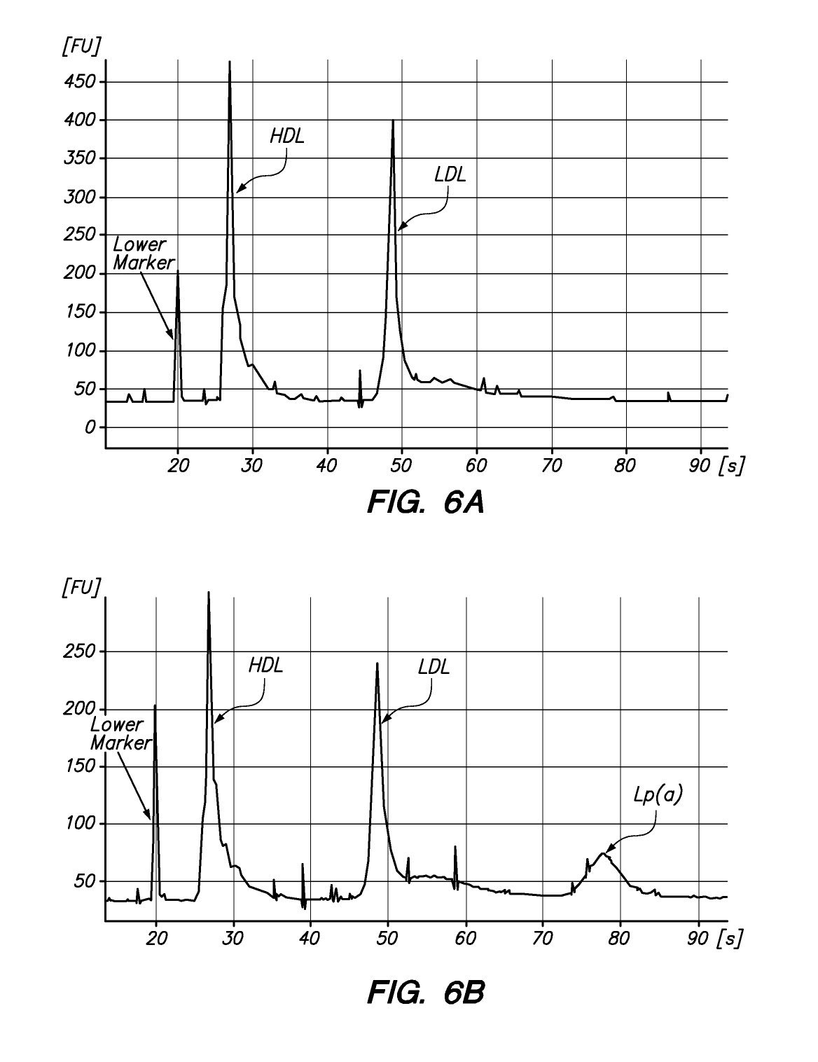

FIG. 6 shows an electropherogram of the samples analyzed on a microfluidic chip with the Bioanalyzer. FIG. 6A shows the analysis of HLD and LDL. The peak due to the alignment marker (“Lower Marker”) is also shown. FIG. 6B shows the analysis of Lp(a).

FIGS. 6A and 6B are composites from different microchannel analyses.

### EXAMPLE 3

In the following, an embodiment of methods and kits according to some embodiments of the present disclosure for the separation of lipoprotein(a) is illustrated, and demonstrates the reproducibility of the Lp(a) analysis.

The sample buffer contained the following reagents:
200 mM TAPS, pH 7.5 (Sigma).
30 μM Vybrant DiD.
1 μM Alexa 700.
0.15 mM SDS (Sigma).
   The separation medium contained the following reagents:
1% Poly(N,N-dimethyl acrylamide) with MW 177 kDa (Polysource, Catalogue no. P6175F4-DMA).
200 mM TAPS, pH 7.5 (Sigma).
0.15 mM SDS (Sigma).
0.15 μM dye V02-04064 (Dyomics) as the dye for focusing the detector.

### Standards:

The Human Lp(a) standard (0.68 mg/mL) (BTI).
The HDL and LDL standards (BTI).
VLDL standard (Sigma).
   The following assay protocol was used:
Take 2 μL of the Lp(a) standard, and mix with 48 μL of sample buffer.
Take 1 μL of serum standard plus 1 μL of Lp(a) standard and mix with 48 μL of sample buffer (Lp(a) spiked sample).
Take 1 μL of the HDL standard, the VLDL standard, and the LDL standard, and separately mix each with 49 μl of sample buffer.
Place chip on primer station.
Label each chip.
Add 10 μL of the separation medium to the matrix well.
Pressurize the well for 1 min.
Fill the other two matrix wells with 10 μL separation medium.
Add 10 μl of diluted serum standard in sample buffer to the standard well.
Add 7 μL of each diluted sample (diluted in sample buffer) to each of the 12 sample wells.
Place chip into instrument and start run.

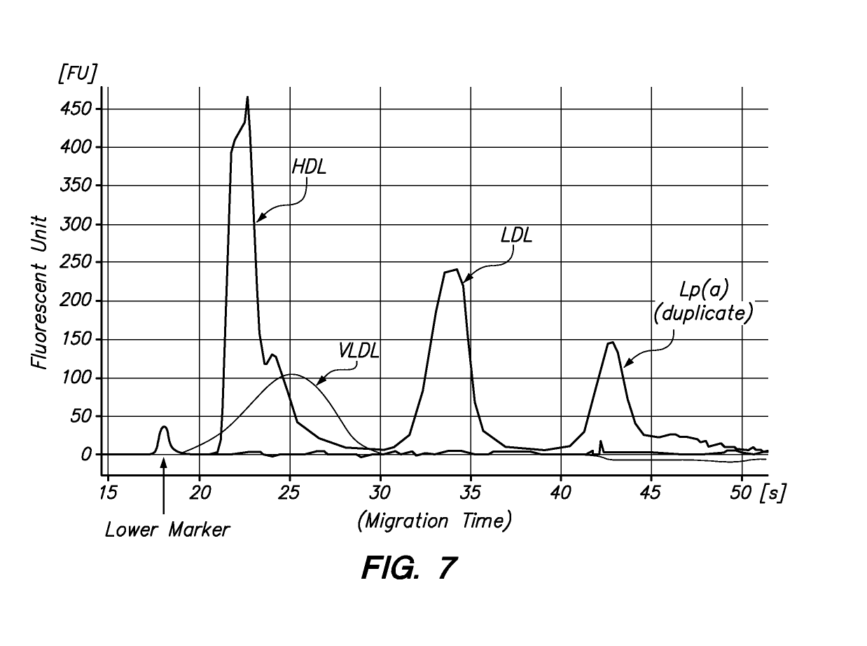

FIG. 7 shows an electropherogram of the samples analyzed on a microfluidic chip with the Bioanalyzer. Two samples containing Lp(a) were injected and gave reproducible peaks which migrated about 40-45 seconds after LDL. FIG. 7 is a composite from different microchannel analyses.

The various embodiments described above are provided by way of illustration only and should not be construed to limit the claims. Those skilled in the art will readily recognize various modifications and changes that can be made without following the example embodiments and applications illustrated and described herein, and without departing from the true spirit and scope of the disclosure or the following claims.

What is claimed is:

1. A method for analyzing lipoprotein(a) in a sample, the method comprising the steps of:
contacting a sample with an associative lipophilic dye;
subjecting the sample to electrophoretic separation through a separation medium;
detecting said dye in said medium, wherein said dye is characterized in that it detectably binds to lipoprotein(a) during said separation and does not detectably bind to albumin or to hemoglobin during said separation.

2. The method of claim 1 wherein said sample comprises serum.

3. The method of claim 1 wherein said contacting comprises contacting the sample with a plurality of different associative lipophilic dyes.

4. The method of claim 1 wherein said associative lipophilic dye comprises a fluorescent dye.

5. The method of claim 1, wherein the medium comprises a non-crosslinked polymer solution.

6. The method of claim 1 wherein said medium comprises poly(N,N-dimethyl acrylamide) having a molecular weight in the range of from 50 kDa to 500 kDa.

7. The method of claim 1 wherein said lipoprotein(a) comprises a size variant of apo(a).

8. The method of claim 1 wherein the separation medium is characterized by having a value of theoretical plates per second (N/s) of at least about 5,
wherein N=5.54×(t/Δt_{1/2})², wherein t is the migration time of a lipoprotein(a) standard,
wherein Δt_{1/2} is the full width at half maximum of a peak due to said standard, and
wherein s is the migration time in seconds.

9. The method of claim 8 wherein the separation medium is characterized by having a N/s value in the range of between 6 and 10.

10. The method of claim 8 wherein said lipoprotein(a) standard has a migration time of less than 60 sec.

11. The method of claim 8 such that said lipoprotein(a) standard can be baseline separated from an LDL standard.

12. The method of claim 11 wherein said lipoprotein(a) migrates at least 10 seconds after said LDL standard.

13. A method of optically detecting lipoprotein(a) in a sample, wherein the method comprises
labelling the lipoprotein(a), with an associative lipophilic dye; and
optically detecting the labelled lipoprotein(a).

14. A kit for optically detecting lipoprotein(a), in a sample, the kit comprising:
a chip for performing a separation of lipoprotein(a), wherein the chip comprises at least one well for receiving a sample, and a separation channel coupled to the at least one well and being adapted for separating different compounds, and
at least one associative lipophilic dye.

15. The kit of claim 14, wherein the separation channel is adapted for separating different compounds electrophoretically, chromatographically or electrochromatographically.

16. The kit of claim 14, comprising separation medium within said separation channel.

17. The kit of claim 16 wherein the separation medium is characterized by having a value of theoretical plates per second (N/s) of at least about 5,
wherein N=5.54×(t/Δt_{1/2})², wherein t is the migration time of a lipoprotein(a) standard,
wherein Δt_{1/2} is the full width at half maximum of a peak due to said standard, and
wherein s is the migration time in seconds.

18. The kit of claim 14, comprising a calibration sample wherein said calibration sample comprises a size variant of lipoprotien(a).

19. The kit according to claim 18, wherein the calibration sample is a “ladder”.

20. The kit of claim 16, wherein said separation medium comprises poly(N,N-dimethyl acrylamide) and a buffer having buffering capacity in an alkaline pH range, wherein the amount of poly(N,N-dimethyl acrylamide) is effective to baseline separate lipoprotein(a) from LDL.

21. A method for selecting a candidate associative lipophilic dye for use in detecting lipoprotein(a), the method comprising:
determining whether said candidate associative lipophilic dye remains detectably bound to albumin and/or to hemoglobin during electrophoretic separation,
determining whether said candidate associative lipophilic dye remains detectably bound to lipoprotein(a) during electrophoretic separation,
wherein a candidate associative lipophilic dye is selected which is characterized in that it detectably binds to lipoprotein(a) during said electrophoretic separation and does not detectably bind to albumin or to hemoglobin during said electrophoretic separation.
